(19) United States
(12) Patent Application Publication
(10) Pub. No.: US 2017/0000745 A1
(43) Pub. Date: Jan. 5, 2017

(54) TRANSDERMAL DRUG DELIVERY SYSTEMS FOR LEVONORGESTREL AND ETHINYL ESTRADIOL

(71) Applicant: Noven Pharmaceuticals, Inc., Miami, FL (US)

(72) Inventors: Joshua Kulakofsky, Miami, FL (US); Jun LIAO, Miami, FL (US)

(73) Assignee: Noven Pharmaceuticals, Inc., Miami, FL (US)

(21) Appl. No.: 15/200,397

(22) Filed: Jul. 1, 2016

Related U.S. Application Data
(60) Provisional application No. 62/188,175, filed on Jul. 2, 2015.

(51) Int. Cl. A61K 9/70; A61K 31/565; A61K 47/34; A61K 47/14; A61K 47/32; A61K 31/567; A61K 47/10

(52) U.S. Cl. CPC A61K 9/7061; A61K 9/7084; A61K 31/567; A61K 31/565; A61K 47/10; A61K 47/14; A61K 47/32; A61K 47/34

(57) ABSTRACT

Described are transdermal drug delivery systems for the transdermal administration of levonorgestrel and ethinyl estradiol, comprising an acrylic polymer matrix. Methods of making and using such systems also are described.

### RELATED APPLICATIONS

This application claims the priority benefits under 35 U.S.C. §119(e) to U.S. provisional application 62/188,175, filed Jul. 2, 2015, the entire contents of which are incorporated herein by reference in their entirety.

### FIELD OF THE INVENTION

Described herein are compositions and methods for the transdermal delivery of levonorgestrel and ethinyl estradiol. The compositions and methods are useful, for example, as contraceptives.

### BACKGROUND

Transdermal delivery systems (adhesive patches) as dosage forms have been the subject of a vast number of patent applications over the last 25 years, yielding many patents but few commercial products in comparison. To those working in the field, the relatively small number of commercial products is not surprising. Although regulatory, economic, and market hurdles play a role in limiting the number of products on the market, the task of developing a transdermal delivery system that achieves desired physical and pharmacokinetic parameters to satisfy physician and patient demand is more daunting. Parameters to be considered during commercial product development may include drug solubility, drug stability (e.g., as may arise from interaction with other component materials and/or the environment), delivery of a therapeutic amount of drug at a desired delivery rate over the intended duration of use, adequate adhesion at the anatomical site of application, integrity (e.g., minimal curling, wrinkling, delaminating and slippage) with minimal discomfort, irritation and sensitization both during use and during and after removal, and minimal residual adhesive (or other components) after removal. Size also may be important from a manufacturing and patient viewpoint, and appearance may be important from a patient viewpoint. These factors become even more complicated when more than one drug is being formulated.

This invention relates generally to transdermal drug delivery systems, and more particularly, to transdermal drug delivery systems for the delivery of levonorgestrel and ethinyl estradiol.

Climara Pro® (Bayer) is a three-layer adhesive-based matrix transdermal patch, comprised of a translucent polyethylene backing film, an acrylate adhesive matrix containing estradiol and levonorgestrel and polyvinylpyrrolidone/vinyl acetate copolymer, and a protective liner of either siliconized or fluoropolymer coated polyester film. The active components are estradiol and levonorgestrel. Climara Pro® is used for the treatment of vasomotor symptoms, which requires therapeutic blood levels only about ⅛^{th} of that required for contraception.

U.S. Pat. No. 7,045,145 is directed to a transdermal delivery system comprising a backing layer, and an adhesive polymer matrix affixed to the backing layer, wherein the adhesive polymer matrix is formulated by combining, on a weight percentage basis: (a) from about 0% to about 10% of a humectant/plasticizer; (b) from about 20% to about 70% of an adhesive copolymer; (c) from about 10% to about 60% percent of a combination of skin permeation enhancing agents which is a mixture comprising dimethyl sulfoxide, a fatty (C8-C20) alcohol ester of lactic acid, a lower (C1-C4) alkyl ester of lactic acid and capric acid present in ratio ranging from about 2:1:1:0.8 to about 6:1:1:0.8, respectively; (d) a progestin hormone; and (e) an estrogen hormone.

U.S. Pat. No. 7,384,650 is directed to a transdermal hormone delivery system comprising a backing layer and an adhesive polymer matrix affixed to the backing layer, wherein the adhesive polymer matrix comprises: (a) an adhesive polymer; (b) a humectant; (c) a combination of skin permeation enhancing agents consisting essentially of, on a final percentage by weight of the adhesive polymer matrix after fabrication of the system, from about 4% to about 12% dimethyl sulfoxide; from about 4.2% to about 12.6% a fatty (C8-C20) alcohol ester of lactic acid; from about 0.7% to about 2.3% lower (C1-C4) alkyl ester of lactic acid; and from about 3% to about 9% capric acid; (d) a progestin; and (e) an estrogen.

U.S. Pat. No. 8,221,785 is directed to a contraceptive delivery system comprising a backing layer and an adhesive polymer matrix affixed to the backing layer, wherein the adhesive polymer matrix comprises: (a) an adhesive polymer comprising a polyacrylate copolymer; (b) a humectant comprising polyvinylpyrrolidone; (c) a combination of skin permeation enhancing agents consisting essentially of, on a final percentage by weight of the adhesive polymer matrix after fabrication of the system, from about 4% to about 12% dimethyl sulfoxide; from about 4.2% to about 12.6% a fatty (C8-C20) alcohol ester of lactic acid; from about 0.7% to about 2.3% lower (C1-C4) alkyl ester of lactic acid; and from about 3% to about 9% capric acid; (d) levonorgestrel; and (e) ethinyl estradiol or 17 beta-estradiol.

U.S. Pat. No. 5,770,219 is directed to a drug-containing matrix for use in a transdermal drug delivery device for administering at least one estrogen to an area of skin or mucosa comprising the estrogen dispersed in a body of a pressure sensitive adhesive, said pressure-sensitive adhesive comprising an acetate acrylate copolymer and polyvinylpyrrolidone, the matrix being essentially free of a skin permeation enhancer. In some embodiments, the matrix further comprises levonorgestrel.

U.S. patent application Ser. No. 14/141,935 is directed to transdermal drug delivery systems for the transdermal delivery of levonorgestrel and ethinyl estradiol in the form of a flexible finite system for topical application, comprising a polymer matrix comprising levonorgestrel, ethinyl estradiol, and an acrylic polymer, wherein the acrylic polymer comprises a hydroxy functional acrylic polymer. In some embodiments, the polymer matrix is substantially free of polyvinylpyrrolidone (PVP) and polyvinylpyrrolidone/vinylacetate (PVP/VA).

Nevertheless, the transdermal delivery of levonorgestrel and ethinyl estradiol continues to present challenges. Some of the challenges presented by this particular drug combination include the high delivery rate of levonorgestrel and its impact on patch size; the undesired crystallization of levonorgestrel in the polymer matrix; and the difficulty of formulating a composition that can achieve sustained drug delivery (e.g., at therapeutic levels) over a period of time of 7 days.

Thus, there remains a need for transdermal drug delivery systems designed for the delivery of specific drugs and drug combinations, such as levonorgestrel and ethinyl estradiol.

### SUMMARY

In accordance with some embodiments, there are provided transdermal drug delivery systems for the transdermal delivery of levonorgestrel and ethinyl estradiol in the form of a flexible finite system for topical application, comprising a polymer matrix comprising levonorgestrel, ethinyl estradiol, an acrylic polymer that comprises a hydroxy functional acrylic polymer, a humectant, a first enhancer selected from the group consisting of glyceryl monooleate, dipropylene glycol, and methyl laurate, a second enhancer different from the first enhancer and selected from the group consisting of glyceryl monooleate, dipropylene glycol, methyl laurate, diethylene glycol monoethyl ether, and dimethyl isosorbide, and, optionally, a third enhancer different from the first and second enhancers and selected from the group consisting of glyceryl monooleate, dipropylene glycol, methyl laurate, diethylene glycol monoethyl ether, and dimethyl isosorbide.

In some embodiments, the first and second enhancers are dipropylene glycol and diethylene glycol monoethyl ether. In some embodiments, the first, second and third enhancers are dipropylene glycol, diethylene glycol monoethyl ether, and glyceryl monooleate. In some embodiments, the first and second enhancers are dipropylene glycol and diethylene glycol monoethyl ether, and the composition further comprises isopropyl myristate as a third enhancer.

In some embodiments, the first penetration enhancer is selected from glyceryl monooleate, dipropylene glycol, and methyl laurate. In some embodiments, the second penetration enhancer is selected from glyceryl monooleate, dipropylene glycol, methyl laurate, diethylene glycol monoethyl ether, and dimethyl isosorbide. In some embodiments, the first penetration enhancer is selected from glyceryl monooleate, dipropylene glycol, and methyl laurate, and the second penetration enhancer is selected from glyceryl monooleate, dipropylene glycol, and methyl laurate, and the polymer matrix optionally further comprises a third penetration enhancer selected from diethylene glycol monoethyl ether and dimethyl isosorbide. In further specific embodiments, the penetration enhancer comprises glyceryl monooleate and dipropylene glycol; glyceryl monooleate and methyl laurate; glyceryl monooleate, dipropylene glycol and diethylene glycol monoethyl ether; glyceryl monooleate, dipropylene glycol and dimethyl isosorbide; glyceryl monooleate, methyl laurate and diethylene glycol monoethyl ether; or glyceryl monooleate, methyl laurate and dimethyl isosorbide.

In accordance with any embodiments, the humectant may be one or more of polyvinylpyrrolidone (PVP), crosslinked PVP (crospovidone) and polyvinylpyrrolidone/vinylacetate (PVP/VA, copovidone).

In accordance with any embodiments, the polymer matrix may comprise on a % dry weight basis about 0.1-3% levonorgestrel, about 0.1-5% ethinyl estradiol, about 1-30% of the first, second and optional third penetration enhancers, about 3-10% humectant, and the balance acrylic polymer(s).

In accordance with any embodiments, the transdermal drug delivery system may comprise an amount of levonorgestrel sufficient to achieve sustained delivery of levonorgestrel over a period of time of at least 3 days, at least 4 days, or at least 7 days.

In accordance with any embodiments, the polymer matrix may further comprise a silicone pressure-sensitive adhesive.

In accordance with any embodiments, the transdermal drug delivery system may comprise an amount of ethinyl estradiol sufficient to achieve sustained delivery of ethinyl estradiol over a period of time of at least 3 days, at least 4 days, or at least 7 days.

In some embodiments, the transdermal drug delivery system further comprises a face adhesive layer disposed on the skin-contacting side of the drug-containing polymer matrix. In particular embodiments, the face adhesive comprises a silicone pressure-sensitive adhesive. In further particular embodiments, the polymer matrix comprises isopropyl myristate as an enhancer, and include a silicone face adhesive.

In any embodiments, the transdermal drug delivery system may further comprise a backing layer and/or a release liner.

Also provided are methods of transdermally delivering levonorgestrel and ethinyl estradiol comprising applying a transdermal drug delivery system as described herein to the skin or mucosa of a subject in need thereof. In some embodiments, the subject is a human female subject. In some embodiments, the method is for contraception. In some embodiments, the transdermal drug delivery system is applied for a duration of up to 7 days.

Also provided are transdermal drug delivery systems as described herein for use in transdermally delivering levonorgestrel and ethinyl estradiol to a subject, and for use to provide contraception to a subject. Also provided are uses of levonorgestrel and ethinyl estradiol in the preparation of transdermal drug delivery system medicaments as described herein, for use in transdermally delivering levonorgestrel and ethinyl estradiol to a subject, and for use in providing contraception to a subject. In accordance with any of these embodiments, the subject may be a human female subject. In accordance with any of these embodiments, the transdermal drug delivery system may be applied for a duration of up to 7 days.

Also provided are methods of preparing a transdermal drug delivery system comprising levonorgestrel and ethinyl estradiol as described herein, comprising preparing a polymer matrix as described herein. In some embodiments, the methods further comprise applying a face adhesive layer to the polymer matrix.

### BRIEF DESCRIPTION OF THE DRAWINGS

FIG. 1A illustrates a monolithic transdermal delivery system as described herein that consists of a backing, a drug-in-adhesive matrix layer, and a release liner (when present). FIG. 1B illustrates a transdermal delivery system as described herein that further comprises a face adhesive layer between the drug-in-adhesive matrix layer and release liner (when present).

FIGS. 2-7 illustrate average and cumulative levonorgestrel (LNG) and ethinyl estradiol (EE) flux (μg/cm²/hr) over time (0-168 hours) from transdermal delivery systems described in the examples as compared to Climara® Pro or OrthoEvra®, respectively. FIG. 2 shows results for systems comprising glyceryl monooleate (GMO) and methyl laurate (MeLau). FIG. 3 shows results for systems comprising glyceryl monooleate (GMO) and dipropylene glycol (DPG). FIG. 4 shows results for systems comprising glyceryl monooleate (GMO) and dipropylene glycol (DPG) and having a face adhesive. FIG. 5 shows results for systems comprising glyceryl monooleate (GMO) and methyl laurate (MeLau) and having a face adhesive. FIG. 6 directly compares results for systems comprising GMO and DPG with and without a face adhesive. FIG. 7 directly compares results for systems comprising GMO and MeLau with and without a face adhesive.

FIG. 8 illustrates average and cumulative levonorgestrel (LNG) and ethinyl estradiol (EE) flux (μg/cm²/hr) over time (0-168 hours) from transdermal delivery systems described in Example 3 (Formulations 20-22) as compared to Climara® Pro or OrthoEvra®, respectively.

FIGS. 9-11 illustrate cumulative and average ethinyl estradiol (EE) (FIGS. 9A, 10A, 11A) and levonorgestrel (LNG) (FIGS. 9B, 10B, 11B) flux (μg/cm²/hr) over time (0-168 hours) from transdermal delivery systems described in Example 4 (Formulations 23-25).

### DETAILED DESCRIPTION

In accordance with some embodiments, the present invention provides transdermal drug delivery systems for the transdermal delivery of levonorgestrel and ethinyl estradiol. In specific embodiments, the systems exhibit desired pharmacokinetic properties, such as by being capable of formulation for use over a 7 day period, and/or exhibit desired physical characteristics, such as satisfactory shear properties, and/or exhibit desired stability characteristics, such as reduced or minimized crystallization of levonorgestrel.

Due to the low solubility of levonorgestrel in pressure-sensitive adhesives, and the low permeability of levonorgestrel, it is difficult to design a transdermal drug delivery system capable of delivering a therapeutically effective amount of levonorgestrel over a period of 7 days from a reasonably sized system.

The present inventors surprisingly discovered that acrylic polymers comprising hydroxy functional groups (such as hydroxyl functional group containing vinyl acetates) are particularly suitable for formulating levonorgestrel for sustained delivery over an extended period of time (such as 7 days). In particular, the inventors determined that such polymers in particular are able to adequately solubilize levonorgestrel (and ethinyl estradiol) while still providing a stable polymer matrix.

The present inventors also surprisingly discovered that each of dipropylene glycol (DPG) and diethylene glycol monoethyl ether (e.g., Transcutol®), alone and in combination with each other and/or with one or more of glyceryl monooleate (GMO) and isopropyl myristate (IMP), are effective enhancers for levonorgestrel. The present inventors also surprisingly discovered that each of dipropylene glycol (DPG), glyceryl monooleate (GMO), and methyl laurate (MeLau), alone and in combination with each other and/or with one or more of diethylene glycol monoethyl ether (Transcutol®) and dimethyl isosorbide (DMI), are effective enhancers for levonorgestrel. They also surprisingly discovered that combinations of dipropylene glycol (DPG) and diethylene glycol monoethyl ether, optionally together with one or more of glyceryl monooleate (GMO) and isopropyl myristate (IMP), are effective enhancers for levonorgestrel, and that combinations of dipropylene glycol (DPG), diethylene glycol monoethyl ether, and glyceryl monooleate (GMO) or combinations of dipropylene glycol (DPG), diethylene glycol monoethyl ether, and isopropyl myristate (IMP) are particularly effective enhancers for levonorgestrel. They also surprisingly discovered that combinations of two or more of DPG, GMO and MeLau are particularly effective enhancers for levonorgestrel, and that combinations of two or more of DPG, GMO and MeLau, together with one or more of diethylene glycol monoethyl ether and DMI also are effective enhancers for levonorgestrel.

The present inventors also surprisingly discovered that including a humectant (such as PVP, crospovidone and/or PVP/VA) in a polymer matrix comprising levonorgestrel is advantageous. For example, the present inventors surprisingly discovered that a humectant can suppress the crystallization of levonogestrel and/or impart desired physical properties, such as the adhesion properties, such as shear.

The present inventors also surprisingly discovered that a face adhesive layer, such as a silicone face adhesive layer, can be used to improve the physical and adhesion properties of the systems described herein without substantially impacting drug flux.

Thus, in accordance with some aspects, there are provided transdermal drug delivery systems and methods for the transdermal delivery of levonorgestrel and ethinyl estradiol. In specific embodiments, the systems exhibit sustained delivery of levonorgestrel and ethinyl estradiol over an extended period of time, such as for at least 3 days, 4 days, 7 days, or longer.

### Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrases “% by weight, based on the dry weight of the polymer matrix” and “% dry weight” refer to the weight of the component(s) in the finished polymer matrix, e.g., after drying and evaporation of volatile processing solvents.

The phrases “% by weight, based on the wet weight of the polymer matrix” and “% wet weight” refer to the weight of the component(s) at issue used to prepare the polymer matrix relative to the weight of the other components in the polymer matrix, not including the weight of the volatile processing solvent(s) used to prepare the polymer matrix.

The phrase “substantially free” as used herein generally means that the described composition (e.g., transdermal drug delivery system, polymer matrix, etc.) comprises less than about 5%, less than about 3%, or less than about 1% by weight, based on the total weight of the composition at issue, of the excluded component. The phrase “free of” as used herein means that the described composition (e.g., polymer matrix, etc.) is formulated without adding the excluded component(s) as an intended component, although trace amounts may be present in other components or as a by-product or contaminant, such that the composition comprises at most only trace amounts of the excluded component(s).

As used herein “subject” denotes any animal in need of drug therapy, including humans. For example, a subject may be suffering from or at risk of developing a condition that can be treated or prevented with levonorgestrel and ethinyl estradiol, or may be taking levonorgestrel and ethinyl estradiol for health maintenance purposes. In specific embodiments, the subject is a female subject taking levonorgestrel and ethinyl estradiol for contraceptive purposes.

As used herein, the phrases “therapeutically effective amount” and “therapeutic level” mean that drug dosage or plasma concentration in a subject, respectively, that provides the specific pharmacological response for which the drug is administered in a subject in need of such treatment. It is emphasized that a therapeutically effective amount or therapeutic level of a drug will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease.

As used herein, “active surface area” means the surface area of the drug-containing layer of the transdermal drug delivery system.

As used herein, “coat weight” refers to the weight of the drug-containing layer per unit area of the active surface area of the transdermal drug delivery system.

As used herein, “flux” (also called “permeation rate”) is defined as the absorption of a drug through skin or mucosal tissue, and is described by Fick's first law of diffusion:

J=−D (dCm/dx)

where J is the flux in g/cm2/sec, D is the diffusion coefficient of the drug through the skin or mucosa in cm2/sec and dCm/dx is the concentration gradient of the drug across the skin or mucosa.

As used herein, the term “transdermal” refers to delivery, administration or application of a drug by means of direct contact with skin or mucosa. Such delivery, administration or application is also known as dermal, percutaneous, transmucosal and buccal. As used herein, “dermal” includes skin and mucosa, which includes oral, buccal, nasal, rectal and vaginal mucosa.

As used herein, “transdermal drug delivery system” refers to a system (e.g., a device) comprising a composition that releases drug upon application to the skin (or any other surface noted above). A transdermal drug delivery system may comprise a drug-containing layer, and, optionally, a backing layer and/or a release liner layer. In some embodiments, the transdermal drug delivery system is a substantially non-aqueous, solid form, capable of conforming to the surface with which it comes into contact, and capable of maintaining such contact so as to facilitate topical application without adverse physiological response, and without being appreciably decomposed by aqueous contact during topical application to a subject. Many such systems are known in the art and commercially available, such as transdermal drug delivery patches. As described below, in one embodiment, the transdermal drug delivery system comprises a drug-containing polymer matrix that comprises a pressure-sensitive adhesive or bioadhesive, and is adopted for direct application to a user's (e.g., a subject's) skin. In other embodiments, the polymer matrix is non-adhesive and may be provided with separate adhesion means (such as a separate adhesive layer) for application and adherence to the user's skin.

As used herein, “polymer matrix” refers to a polymer composition which contains one or more drugs. In some embodiments, the matrix comprises a pressure-sensitive adhesive polymer or a bioadhesive polymer. In other embodiments, the matrix does not comprise a pressure-sensitive adhesive or bioadhesive. As used herein, a polymer is an “adhesive” if it has the properties of an adhesive per se, or if it functions as an adhesive by the addition of tackifiers, plasticizers, crosslinking agents or other additives. Thus, in some embodiments, the polymer matrix comprises a pressure-sensitive adhesive polymer or a bioadhesive polymer, with drug dissolved or dispersed therein. The polymer matrix also may comprise tackifiers, plasticizers, crosslinking agents, enhancers, co-solvents, fillers, antioxidants, solubilizers, crystallization inhibitors, or other additives described herein. U.S. Pat. No. 6,024,976 describes polymer blends that are useful in accordance with the transdermal systems described herein. The entire contents of U.S. Pat. No. 6,024,976 is incorporated herein by reference.

As used herein, the term “pressure-sensitive adhesive” refers to a viscoelastic material which adheres instantaneously to most substrates with the application of very slight pressure and remains permanently tacky. A polymer is a pressure-sensitive adhesive within the meaning of the term as used herein if it has the properties of a pressure-sensitive adhesive per se or functions as a pressure-sensitive adhesive by admixture with tackifiers, plasticizers or other additives.

The term pressure-sensitive adhesive also includes mixtures of different polymers and mixtures of polymers, such as polyisobutylenes (PIB), of different molecular weights, wherein each resultant mixture is a pressure-sensitive adhesive. In the last case, the polymers of lower molecular weight in the mixture are not considered to be “tackifiers,” said term being reserved for additives which differ other than in molecular weight from the polymers to which they are added.

In some embodiments, the polymer matrix is a pressure-sensitive adhesive at room temperature and has other desirable characteristics for adhesives used in the transdermal drug delivery art. Such characteristics include good adherence to skin, ability to be peeled or otherwise removed without substantial trauma to the skin, retention of tack with aging, etc. In some embodiments, the polymer matrix has a glass transition temperature (Tg), measured using a differential scanning calorimeter, of between about −70° C. and 0° C.

As used herein, the term “rubber-based pressure-sensitive adhesive” refers to a viscoelastic material which has the properties of a pressure-sensitive adhesive and which contains at least one natural or synthetic elastomeric polymer.

In some embodiments, the transdermal drug delivery system includes one or more additional layers, such as one or more additional polymer matrix layers, or one or more adhesive layers that adhere the transdermal drug delivery system to the user's skin. In specific embodiments, the transdermal drug delivery system includes a face adhesive layer disposed on the skin-contacting side of the drug-containing polymer matrix. In other embodiments, the transdermal drug delivery system is monolithic, meaning that it comprises a single polymer matrix layer comprising a pressure-sensitive adhesive or bioadhesive with drug dissolved or dispersed therein, and no rate-controlling membrane or face adhesive.

The transdermal drug delivery system also may include a drug impermeable backing layer or film. In some embodiments, the backing layer is adjacent one face of the polymer matrix layer. When present, the backing layer protects the polymer matrix layer (and any other layers present) from the environment and prevents loss of the drug and/or release of other components to the environment during use. Materials suitable for use as backing layers are well-known known in the art and can comprise films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. For example, 3M's Scotch Pak™ 1012 or 9732 backing material (a polyester film with an ethylene vinyl acetate copolymer heat seal layer) is useful in the transdermal drug delivery systems described herein.

The transdermal drug delivery system also may include a release liner, typically located adjacent the opposite face of the system as compared to the backing layer. When present, the release liner is removed from the system prior to use to expose the polymer matrix layer and/or an adhesive layer prior to topical application. Materials suitable for use as release liners are well-known known in the art and include the commercially available products of Dow Coming Corporation designated Bio-Release® liner and Syl-off® 7610 (both silicone-based) and 3M's 1020, 1022, 9744, 9748 and 9749 Scotchpak™ (fluoropolymer coated polyester films).

The transdermal drug delivery system may be packaged or provided in a package, such as a pouchstock material used in the prior art for transdermal drug delivery systems in general. For example, DuPont's Surlyn® can be used in a pouchstock material.

As used herein, a “monolithic” transdermal drug delivery system may include a backing layer and/or release liner, and may be provided in a package.

Polymer Matrix

In accordance with some embodiments, the compositions described herein comprise a polymer matrix that comprises, consists essentially of, or consists of levonorgestrel, ethinyl estradiol, at least one acrylic polymer, at least one penetration enhancer, and a humectant. In this context, the phrase “consists essentially of” means that the polymer matrix is substantially free of other polymer components (e.g., substantially free of polymers other than one or more acrylic polymers) although it may include other excipients known to be useful in transdermal compositions (such as tackifiers, plasticizers, crosslinking agents or other excipients known in the art) as long as those other excipients do not degrade the physical and/or pharmacokinetic properties of the compositions to pharmaceutically unacceptable levels.

In some embodiments, the polymer matrix comprises (on a % dry weight basis) about 0.1-3% levonorgestrel, about 0.1-5% ethinyl estradiol, about 1-30% penetration enhancer(s), about 3-10% humectant, and the balance acrylic polymer(s).

Levonorgestrel

Levonorgestrel is a synthetic progestogen. It is an enantiomer of the chiral compound 13-ethyl-17-ethynyl-17-hydroxy-1,2,6,7,8,9,10,11,12,13,14,15,16,17-tetradecahydrocyclopenta[a]phenanthren-3-one.

Ethinyl Estradiol

Ethinyl estradiol is an estrogen with the chemical name 19-nor-17α-pregna-1,3,5(10)-trien-20-yne-3,17-diol.

The amount of levonorgestrel and ethinyl estradiol to be incorporated in the polymer matrix varies depending on the desired therapeutic effect, and the time span for which the system is to provide therapy. For most drugs, the passage of the drugs through the skin will be the rate-limiting step in delivery. A minimum amount of drug in the system is selected based on the amount of drug which passes through the skin in the time span for which the system is to provide therapy. In some embodiments, a system for the transdermal delivery of levonorgestrel and ethinyl estradiolis used over a period of about 1 day, about 3 days, about 7 days, or longer. Thus, in one embodiment, the systems comprise an amount of drug (e.g., levonorgestrel and ethinyl estradiol) sufficient to deliver therapeutically effective amounts of drug over a period of from 1 day to 3 days, 7 days, or longer, including for 1 day, for 2 days, for 3 days, for 4 days, for 5 days, for 6 days, for 7 days, or for longer.

The inventors surprisingly discovered that the amount of ethinyl estradiol can be selected and controlled to select and control the transdermal delivery of ethinyl estradiol without substantially impacting the transdermal delivery of levonorgestrel. That is, the flux of ethinyl estradiol can be increased by increasing the relative amount of ethinyl estradiol without substantially impacting the transdermal delivery of levonorgestrel.

In some embodiments, the polymer matrix comprises from about 0.1% to about 50% dry weight, including from about 1% to about 20%, such as from about 1% to about 10% dry weight, of active agent. In some embodiments, the polymer matrix comprises from about 0.1% to about 25% dry weight, including from about 1% to about 10%, such as from about 1% to about 5% dry weight, of levonorgestrel. In some embodiments, the polymer matrix comprises from about 0.1% to about 25% dry weight, including from about 1% to about 10%, such as from about 1% to about 5% dry weight, of ethinyl estradiol.

In some embodiments, the polymer matrix comprises from about 0.1 to 3% dry weight or 0.1 to 5% dry weight levonorgestrel. In some embodiments, the polymer matrix comprises from about 0.1 to 3% dry weight or 0.1 to 5% dry weight ethinyl estradiol.

Acrylic Polymers

The term “acrylic polymer” is used here as in the art interchangeably with “polyacrylate,” “polyacrylic polymer,” and “acrylic adhesive.” The acrylic-based polymers can be any of the homopolymers, copolymers, terpolymers, and the like of various acrylic acids or esters. In some embodiments, the acrylic-based polymers are adhesive polymers. In other embodiments, the acrylic-based polymers function as an adhesive by the addition of tackifiers, plasticizers, crosslinking agents or other additives.

The acrylic polymer can include copolymers, terpolymers and multipolymers. For example, the acrylic polymer can be any of the homopolymers, copolymers, terpolymers, and the like of various acrylic acids. In some embodiments, the amount and type of acrylic polymer is dependent on the amounts of levonorgestrel and ethinyl estradiol used.

Acrylic polymers useful in practicing the invention include polymers of one or more monomers of acrylic acids and other copolymerizable monomers. The acrylic polymers also include copolymers of alkyl acrylates and/or methacrylates and/or copolymerizable secondary monomers or monomers with functional groups. Combinations of acrylic-based polymers based on their functional groups is also contemplated. Acrylic-based polymers having functional groups include copolymers and terpolymers which contain, in addition to nonfunctional monomer units, further monomer units having free functional groups. The monomers can be monofunctional or polyfunctional. By varying the amount of each type of monomer added, the cohesive properties of the resulting acrylic polymer can be changed as is known in the art. In some embodiments, the acrylic polymer is composed of at least 50% by weight of an acrylate or alkyl acrylate monomer, from 0 to 20% of a functional monomer copolymerizable with the acrylate, and from 0 to 40% of other monomers.

Acrylate monomers which can be used include acrylic acid and methacrylic acid and alkyl acrylic or methacrylic esters such as methyl acrylate, ethyl acrylate, propyl acrylate, amyl acrylate, butyl acrylate, butyl methacrylate, hexyl acrylate, methyl methacrylate, hexyl methacrylate, heptyl acrylate, octyl acrylate, nonyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, glycidyl acrylate, and corresponding methacrylic esters.

Non-functional acrylic-based polymers can include any acrylic based polymer having no or substantially no free functional groups.

Functional monomers, copolymerizable with the above alkyl acrylates or methacrylates, which can be used include acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate.

As used herein, “functional monomers or groups,” are monomer units typically in acrylic-based polymers which have reactive chemical groups which modify the acrylic-based polymers directly or which provide sites for further reactions. Examples of functional groups include carboxyl, epoxy, hydroxyl, sulfoxyl, and amino groups. Acrylic-based polymers having functional groups contain, in addition to the nonfunctional monomer units described above, further monomer units having free functional groups. The monomers can be monofunctional or polyfunctional. These functional groups include carboxyl groups, hydroxy groups, amino groups, amido groups, epoxy groups, etc. Typical carboxyl functional monomers include acrylic acid, methacrylic acid, itaconic acid, maleic acid, and crotonic acid. Typical hydroxy functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate. As noted above, in some embodiments, the acrylic polymer does not include such functional groups.

In some embodiments, the acrylic polymer includes hydroxy functional monomers, including acrylic polymers having vinyl acetate groups. Such polymers generally exhibit good solubility for levonorgestrel, which allows sufficient loading of levonorgestrel for preparation of a system that achieves transdermal delivery of a therapeutically effective amount of active agent over an extended period of time, such as a period of at least 3 days, at least 4 days, or at least 7 days, or longer.

Further details and examples of acrylic adhesives which are suitable in the practice of the invention are described in Satas, “Acrylic Adhesives,” Handbook of Pressure-Sensitive Adhesive Technology, 2nd ed., pp. 396-456 (D. Satas, ed.), Van Nostrand Reinhold, New York (1989); “Acrylic and Methacrylic Ester Polymers,” Polymer Science and Engineering, Vol. 1, 2nd ed., pp 234-268, John Wiley & Sons, (1984); U.S. Pat. No. 4,390,520; and U.S. Pat. No. 4,994,267, all of which are expressly incorporated by reference in their entireties.

Suitable acrylic polymers also include pressure-sensitive adhesives which are commercially available, such as the acrylic-based adhesives sold under the trademarks DURO-TAK® (such as DURO-TAK® 87-900A, 87-2516, 87-2287, -4098, -4287, -2852, -2196, -2296, -2194, -2516, -2070, -2353, -2154, -2510, -9085, -9088 and 73-9301) and GELVA® Multipolymer Solution (such as GELVA® 2480, 788, 737, 263, 1430, 1753, 1151, 2450, 2495, 3067, 3071, 3087 and 3235) both by Henkel Corporation, Bridgewater, N.J. Other suitable acrylic adhesives include those sold under the trademark EUDRAGIT® by Evonik Industries AG Pharma Polymers, Darmstadt, Germany. For example, hydroxy functional adhesives with a reactive functional OH group in the polymeric chain, can be used. Non-limiting commercial examples of this type of adhesives include both GELVA® 737, 788, and 1151, and DURO-TAK® 87-2287, -4287, -2510, -2516 387-2510, and 387-2287.

In some embodiments, the acrylic polymer constitutes up to 100% by weight of the polymer content of the polymer matrix, including 100%

Silicone Polymers

The term “silicone polymer” is used herein interchangeably with the terms siloxane, polysiloxane, and silicones as known in the art. A suitable silicone polymer may also be a pressure-sensitive adhesive. Thus, in some embodiments, the silicone-based polymer is an adhesive polymer. In other embodiments, the silicone-based polymer functions as an adhesive by the addition of tackifiers, plasticizers, crosslinking agents, or other additives.

Suitable silicone polymers include silicone pressure-sensitive adhesives which are based on two major components: (i) a polymer or gum and (ii) a tackifying resin. A silicone pressure-sensitive adhesive can be prepared by cross-linking a gum, typically a high molecular weight polydiorganosiloxane, with a resin, to produce a three-dimensional silicate structure, via a condensation reaction in an appropriate organic, volatile solvent, such as ethyl acetate or heptane. The ratio of resin to polymer can be adjusted in order to modify the physical properties of silicone pressure-sensitive adhesive. See Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, New York (1989).

Exemplary silicone pressure-sensitive polymers are adhesives (e.g., capable of sticking to the site of topical application), including pressure-sensitive adhesives. Illustrative examples of silicone polymers having reduced silanol concentrations include silicone-based adhesives (and capped polysiloxane adhesives) such as those described in U.S. Pat. No. Re. 35,474 and U.S. Pat. No. 6,337,086, which are incorporated herein by reference in their entireties, and which are commercially available from Dow Corning Corporation (Dow Corning Corporation, Medical Products, Midland, Mich.) as BIO-PSA® 7-4100, -4200 and -4300 product series, and non-sensitizing, pressure-sensitive adhesives produced with compatible organic volatile solvents (such as ethyl acetate or heptane) and available commercially under their BIO-PSA® 7-4400 series, -4500 series and -4600 series.

Further details and examples of silicone pressure-sensitive adhesives which are useful in the polymer matrices and compositions and methods described herein are mentioned in the following U.S. Pat. Nos. 4,591,622; 4,584,355; 4,585,836; and 4,655,767, which are all expressly incorporated by reference herein in their entireties. It should also be understood that silicone fluids are also contemplated for use in the polymer matrices and methods described herein.

In some embodiments, the silicone polymer constitutes from about 1% to about 25% of the polymer matrix, based on the dry weight of the polymer matrix, including about 1%, about 5%, about 10%, about 15%, or about 25% by weight. In some embodiments, the amount and type of silicone polymer is dependent on the amount and type of acrylic polymer used in the polymer matrix (if any) and/or the amount of levonorgestrel and estradiol being formulated. For example, in some embodiments, the amount of acrylic polymer and silicone polymer are adjusted to modify the saturation concentration of the drugs in the polymer matrix in order to affect the rate of delivery of the drugs from the system and through the skin.

Penetration Enhancers

In some embodiments, the polymer matrix comprises a penetration enhancer. A “penetration enhancer” is an agent known to accelerate the delivery of the drug through the skin. These agents also have been referred to as accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyidecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

As noted above, one problem addressed by the present invention is the low skin permeability of levonorgestrel. The present inventors surprisingly discovered that each of dipropylene glycol (DPG) and diethylene glycol monoethyl ether, alone and in combination with each other and/or with one or more of glyceryl monooleate (GMO) and isopropyl myristate (IMP), are effective enhancers for levonorgestrel. They also surprisingly discovered that combinations of dipropylene glycol (DPG) and diethylene glycol monoethyl ether, optionally together with one or more of glyceryl monooleate (GMO) and isopropyl myristate (IMP), are effective enhancers for levonorgestrel, and that combinations of dipropylene glycol (DPG), diethylene glycol monoethyl ether, and glyceryl monooleate (GMO) or combinations of dipropylene glycol (DPG), diethylene glycol monoethyl ether, and isopropyl myristate (IMP) are particularly effective enhancers for levonorgestrel. Thus, in some embodiments, the penetration enhancer comprises DPG and diethylene glycol monoethyl ether (Transcutol®), optionally with one or more of GMO and IPM, including combinations of DPG, diethylene glycol monoethyl ether and GMO and combinations of DPG, diethylene glycol monoethyl ether and IPM.

The present inventors also surprisingly discovered that each of DPG, GMO and MeLau, alone and in combination with each other and/or with one or more of diethylene glycol monoethyl ether and/or DMI, are effective enhancers for levonorgestrel, and that combinations of two or more of DPG, GMO and MeLau, alone or in further combination with one or more of diethylene glycol monoethyl ether and/or DMI, are particularly effective. Thus, in some embodiments, the penetration enhancer comprises DPG, GMO and MeLau, or any two or more thereof, including all three. In other embodiments, the penetration enhancer comprises a mixture of one or more of GMO, DPG and MeLau and one or more of diethylene glycol monoethyl ether and DMI. In specific embodiments, the penetration enhancer comprises glyceryl monooleate and dipropylene glycol; glyceryl monooleate and methyl laurate; glyceryl monooleate, dipropylene glycol and diethylene glycol monoethyl ether; or glyceryl monooleate, dipropylene glycol and dimethyl isosorbide.

It was surprisingly found that the use of these enhancers resulted in enhanced levonorgestrel flux effective for contraception over a sustained delivery period, such as 7 days. These effects are discussed in more detail in the examples below. (Because ethinyl estradiol has a relatively high solubility in hydroxyl functional acrylic polymers, its flux can be adjusted by modifying the its concentration in the polymer matrix.)

In some embodiments, the total amount of penetration enhancer(s) is up to about 30% dry weight of the polymer matrix, including up to 30%, up to about 20%, including 20%, up to about 10%, including 10%, or up to about 5%, including 5% by dry weight. In some embodiments, the total amount of penetration enhancer(s) is from about 1% to about 25%, including about 1%, 2%, 3%, 4%, 5%, 6%, 10%, 15%, 20%, 25%, 28% or 30% by weight.

When a combination of enhancers is used, individual enhancers may be used in varying amounts, such as about 0.05%, 0.1%, 0.15%, 0.2%, 0.25%, 0.3%, 0.35%, 0.4%, 0.45%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 11.0%, 12.0%, 13.0%, 14.0%, 15.0%, 16.0%, 17.05%, 18.0%, 19.0% and 20.0% by weight, based on the dry weight of the polymer matrix. For example, DPG can enhance the flux of levonorgestrel when used in an amount of about 5% dry weight or less, or about 2.75% by dry weight or less. GMO can enhance the flux of levonorgestrel when used in an amount of about 6.5% by dry weight or less, or about 3% by dry weight or less. Diethylene glycol monoethyl ether can enhance the flux of levonorgestrel when used in an amount of about 0.5% by dry weight or less, about 0.4% by dry weight or less, or about 0.3% by dry weight or less. DMI can enhance the flux of levonorgestrel when used in an amount of about 1.5% by dry weight or less. IPM can enhance the flux of levonorgestrel when used in an amount of about 5% by dry weight or less. Given this guidance, a person of ordinary skill in this field can determine flux-enhancing amounts of each individual enhancer described herein used in combination with one or more other enhancers described herein by routine screening, such as by assessing the flux of levonorgestrel with varying amounts of the enhancer(s).

In some embodiments, the polymer matrix comprises about 2.75% by weight dipropylene glycol and from about 0.3% to about 0.4% by weight diethylene glycol monoethyl ether (including about 0.3% or about 0.4% by weight diethylene glycol monoethyl ether) and from about 3% to about 6.5% by weight glyceryl monooleate (including about 3% or about 6.5% by weight glyceryl monooleate). In other embodiments, the polymer matrix comprises about 5% by weight dipropylene glycol and from about 0.3% to about 0.4% by weight diethylene glycol monoethyl ether (including about 0.3% or about 0.4% by weight diethylene glycol monoethyl ether), and about 5% by weight isopropyl myristate, all based on the dry weight of the polymer matrix (% wet dry).

In some embodiments, the polymer matrix comprises about 10% by weight glyceryl monooleate and 10% by weight dipropylene glycol; or about 10% by weight glyceryl monooleate and about 5% by weight methyl laurate; or about 10% by weight glyceryl monooleate, about 10% by weight dipropylene glycol and about 8% by weight diethylene glycol monoethyl ether; or about 10% by weight glyceryl monooleate, about 10% by weight dipropylene glycol and about 8% by weight dimethyl isosorbide, all based on the wet weight of the polymer matrix (% wet weight).

In other embodiments, the polymer matrix comprises about 3% by weight glyceryl monooleate, about 6.5% by weight dipropylene glycol and about 2.1% by weight diethylene glycol monoethyl ether; or about 3% by weight glyceryl monooleate, about 4.2% by weight dipropylene glycol and about 2.75% by weight diethylene glycol monoethyl ether; or about 3% by weight glyceryl monooleate, about 4.2% by weight dipropylene glycol and about 4.2% by weight dimethyl isosorbide, all based on the wet weight of the polymer matrix (% wet weight).

In other embodiments, the polymer matrix comprises about 3% by weight glyceryl monooleate, about 4.25% by weight dipropylene glycol and about 0.3% by weight diethylene glycol monoethyl ether; or about 3% by weight glyceryl monooleate, about 2.75% by weight dipropylene glycol and about 0.4% by weight diethylene glycol monoethyl ether; or about 3% by weight glyceryl monooleate, about 2.75% by weight dipropylene glycol and about 2.5% by weight dimethyl isosorbide, all based on the dry weight of the polymer matrix (% dry weight).

Humectant

In some embodiments, the polymer matrix comprises a humectant. Humectants suitable for use in transdermal drug delivery systems are known, and include polyvinylpyrrolidone (PVP), crosslinked PVP (crospovidone) and polyvinylpyrrolidone/vinylacetate (PVP/VA, copovidone), and combinations of any two or more thereof

The amount of humectant can be selected based on desired properties, such as an amount effective to suppress the crystallization of levonogestrel and/or an amount effective to impart desired physical properties, such as the adhesion properties of the polymer matrix, such as shear. In some embodiments, a humectant is used in an amount up from about 3% to about 10% dry weight of the polymer matrix, including about 3%, about 5% or about 10% dry weight.

Other Components

The polymer matrix may further comprise various thickeners, fillers, and other additives or components known for use in transdermal drug delivery systems.

As noted above, in embodiments where the polymer matrix comprises a pressure-sensitive adhesive or bioadhesive, the polymer matrix can serve as an adhesive portion of the system (e.g., a reservoir device), or can serve as one or more layers of a multi-layer system. Alternatively, a polymer matrix comprising a pressure-sensitive adhesive or bioadhesive with drug dissolved or dispersed therein can constitute a monolithic device. In embodiments where the polymer matrix does not comprise an adhesive, but instead, for example, comprises a polymeric drug reservoir, it can be used in combination with one or more adhesive layers, or with a surrounding adhesive portion, as is well known to those skilled in the art.

Transdermal Drug Delivery Systems

In some embodiments, the transdermal drug delivery system consists essentially of the polymer matrix layer. By “consists essentially of the polymer matrix layer” means that the system does not contain any other layers that affect drug delivery, such as an additional face adhesive layer, rate-controlling polymer layer, rate-controlling membrane, or drug reservoir layer. It will be understood, however, that the system that consists essentially of the polymer matrix layer may comprise a backing layer and/or release liner.

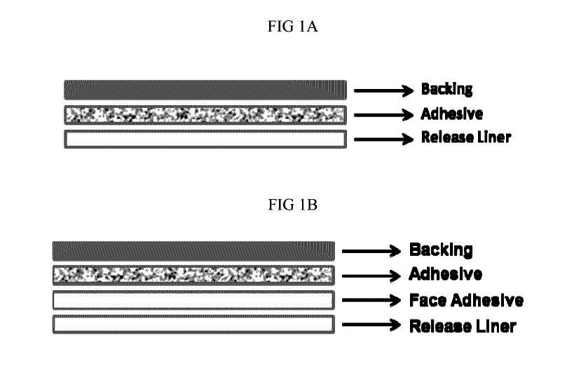

In some embodiments, the transdermal drug delivery system includes a face adhesive layer disposed on the skin-contacting side of the drug-containing polymer layer, as illustrated in FIG. 1B. In specific embodiments, the face adhesive is a silicone face adhesive comprising a silicone adhesive, such as a silicone pressure-sensitive adhesive and, optionally, one or more penetration enhancers, such as those discussed above. In specific embodiments, the face adhesive comprises, on a dry weight/weight basis, from about 90 to about 100% of a silicone adhesive polymer and from about 0 to about 10% of one or more penetration enhancers. Suitable pressure sensitive adhesives for use in the face adhesive layer include silicone pressure sensitive adhesives manufactured by Dow Coming, such as those in the BIO-PSA 4100, 4200, 4300, 4500, 4600 series. A face adhesive may be comprised of a single silicone pressure sensitive adhesives or a blend of two or more thereof. In specific embodiments, the face adhesive is comprised of Bio PSA 4502, or a blend of BIO-PSA 5402 and 4602, such as a blend having a 1:1 ratio or 1:3 ratio of BIO-PSA 4502:Bio-PSA 4602. As noted above and illustrated in the examples below, it was surprisingly discovered that the presence of a face adhesive layer in the systems described herein does not substantially impact drug flux.

The system may be of any shape or size suitable for transdermal application. The polymer matrices described herein may be prepared by methods known in the art. The polymer matrices can be formed into systems by methods known in the art. For example, the polymer matrix material can be applied to a backing layer and release liner by methods known in the art, and formed into sizes and shapes suitable for use. For example, after the polymer matrix is formed, it may be brought into contact with a support layer, such a releaser liner layer or backing layer, in any manner known to those of skill in the art. Such techniques include calender coating, hot melt coating, solution coating, etc. When a face adhesive layer is present, the system may be made by forming the polymer matrix on a backing material, forming the face adhesive layer on a release liner, and then applying the face adhesive layer to the polymer matrix layer.

For example, a polymer matrix can be prepared by blending the components of the polymer matrix, applying the matrix material to a support layer such as a backing layer or release liner, and removing any remaining solvents, such as by drying. The levonorgestrel and ethinyl estradiol can be added at any stage. In one embodiment, all polymer matrix components, including levonorgestrel and ethinyl estradiol, are blended together. In another embodiment, the polymer matrix components other than levonorgestrel and ethinyl estradiol are blended together, and then the levonorgestrel and ethinyl estradiol is dissolved or dispersed therein. The order of steps, amount of ingredients, and the amount and time of agitation or mixing can be determined and optimized by the skilled practitioner. An exemplary general method is as follows:

Appropriate amounts of polymer(s), levonorgestrel, ethinyl estradiol, enhancer(s), and organic solvent(s) (for example toluene, or ethyl acetate an/or isopropyl alcohol) are combined and thoroughly mixed together in a vessel.

The formulation is then transferred to a coating operation where it is coated onto a protective release liner at a controlled specified thickness. The coated product is then passed through an oven in order to drive off all volatile processing solvents.

The dried product on the release liner is then joined to the backing material and wound into rolls for storage.

When a face adhesive is used, a face adhesive solution containing the face adhesive in a suitable solvent may be coated onto a release liner and dried in a convection oven. The dried face adhesive on the release liner may be laminated with the prepared drug-in-adhesive polymer matrix (after removing its release liner) to form a laminate with a face adhesive.

Appropriate size and shape “systems” are die-cut from the roll material and then pouched.

Other manufacturing methods are known in the art that are suitable for making the systems described herein.

In some embodiments, there is provided a method of effecting transdermal drug delivery of levonorgestrel and ethinyl estradiol, by applying a system as described herein to the skin or mucosa of a subject in need thereof. In some embodiments, the system is applied over a period of at least about 1 day, at least about 2 days, at least about 3 days, at least about 4 days, at least about 5 days, at least about 6 days, or at least about 7 days, such as for 1, 2, 3, 4, 5, 6 or 7 days. In some embodiments, the method is effective to achieve therapeutic levels of levonorgestrel and ethinyl estradiol in the subject during the application period.

In some embodiments, the systems described herein are designed for use by female patients, such as for contraception.

The following specific examples are included as illustrative of the transdermal drug delivery systems and polymer matrices described herein. These examples are in no way intended to limit the scope of the invention. Other aspects of the invention will be apparent to those skilled in the art to which the invention pertains.

### EXAMPLE 1

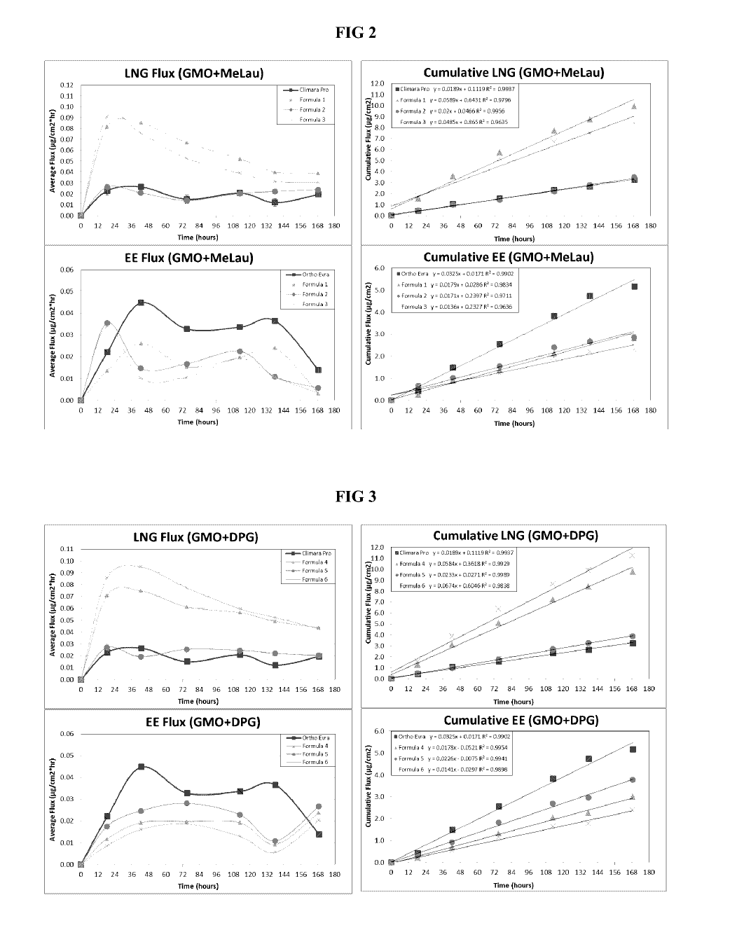

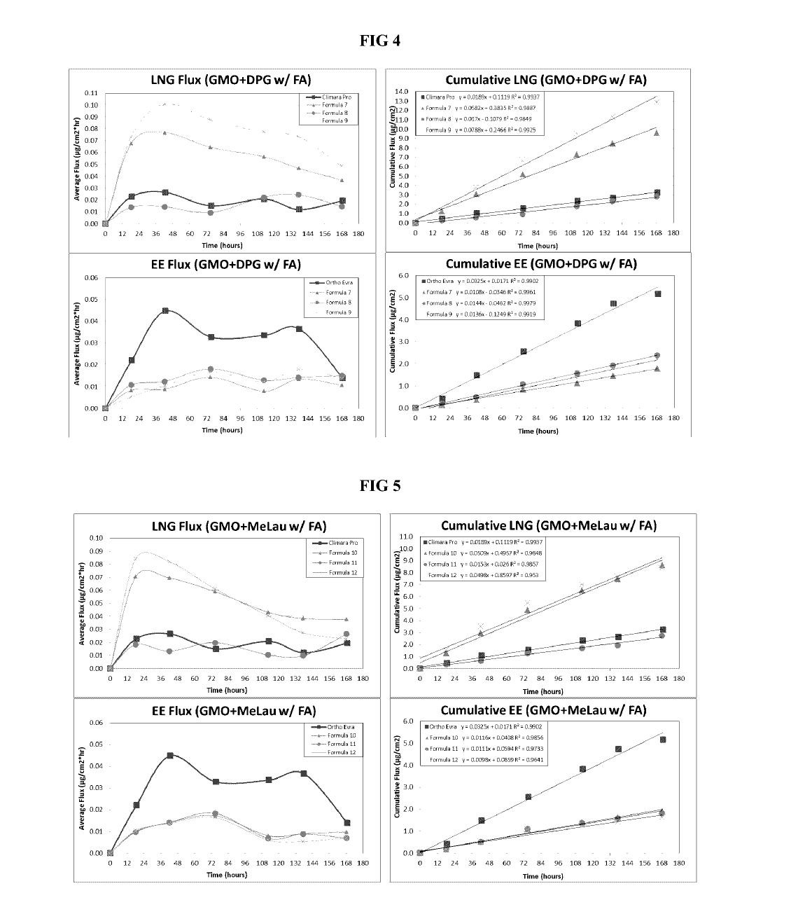

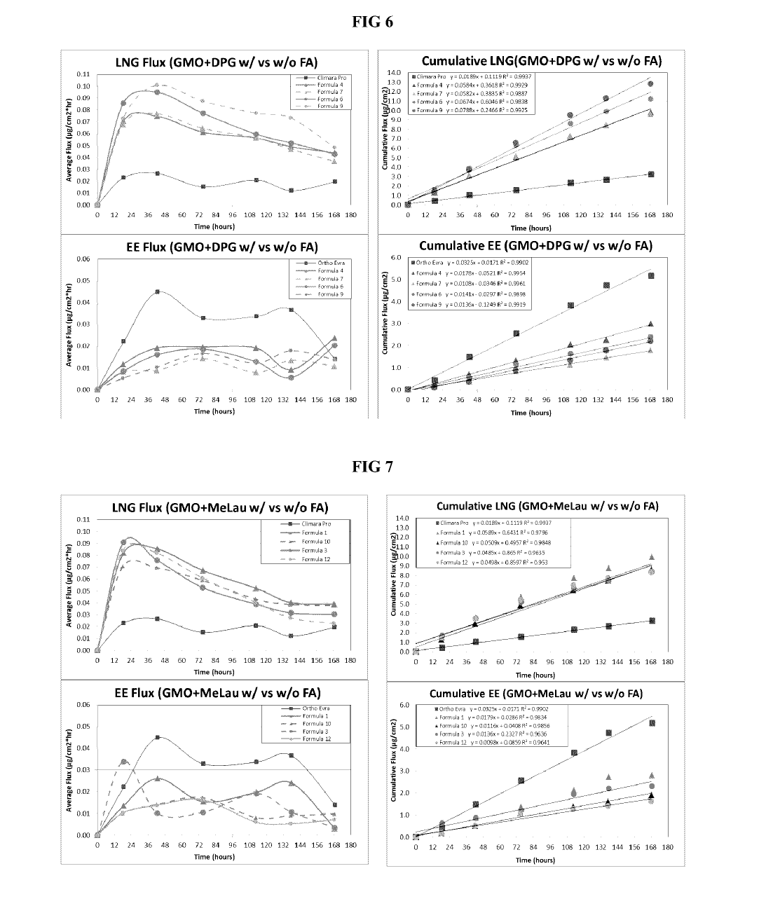

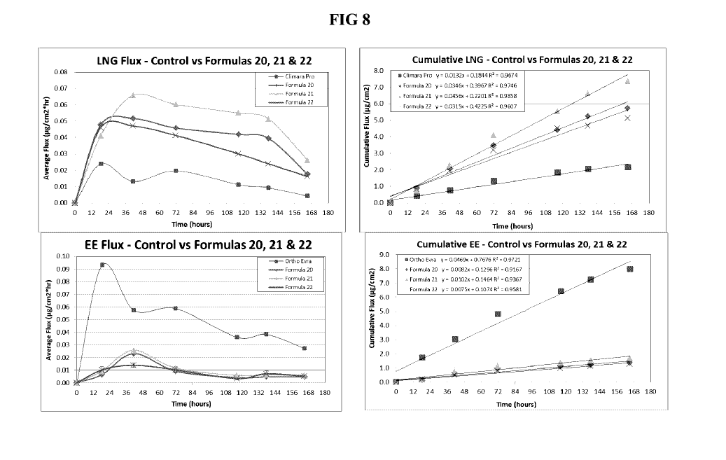

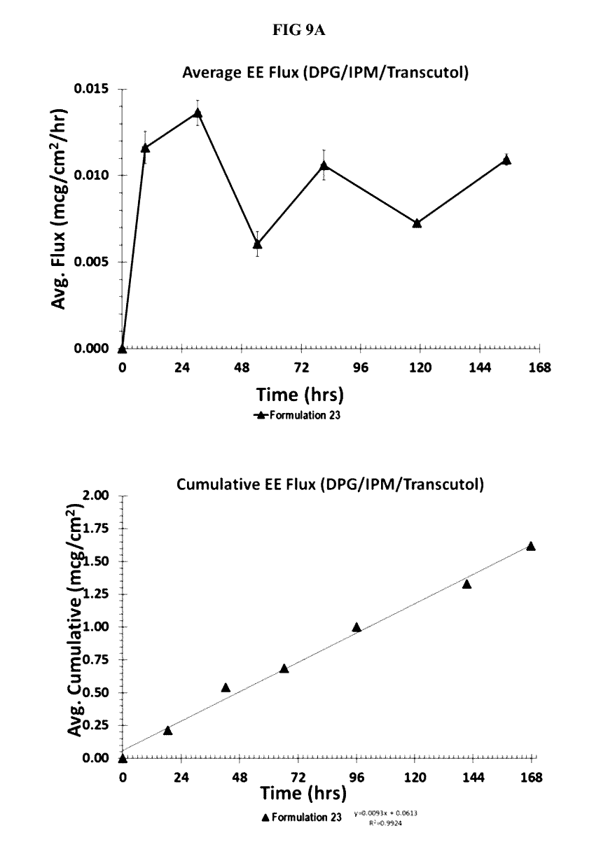

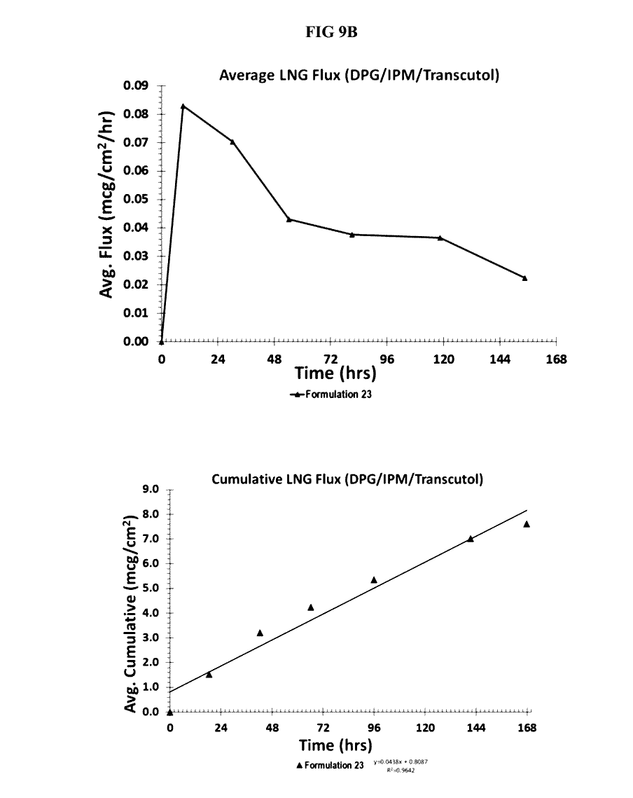

Polymer matrices comprising 0.5% ethinyl estradiol and 1.2% levonogestrel in a hydroxyl-functional acrylic polymer were prepared with the following components, applied to a backing material, dried, and optionally provided a face adhesive layer comprising a blend of silicone pressure-sensitive adhesives. The shear properties and drug flux through human cadaver skin were assessed. Shear results are set forth in the table below, while flux results are set forth in FIGS. 2-5, as compared to Climara® Pro (levonogestrel) or Ortho Evra (ethinyl estradiol). FIGS. 6 and 7 directly compare drug flux using systems with and without a face adhesive.

| Formulation | | | | |
|---|---|---|---|---|
| (wt % | | | | |
| acrylic | | | Face | Avg. Shear |
| polymer) | Enhancers | Humectant | Adhesive | (Std. Dev.) |
| 1 | GMO 10% | (none) | (none) | 32.4 |
| (83.3) | MeLau 5% | | | (0.8) |
| 2 | GMO 10% | 10% | (none) | 68.9 |
| (73.3) | MeLau 5% | crospovidone | | (2.6) |
| 3 | GMO 10% | 10% PVP | (none) | 184.2 |
| (73.3) | MeLau 5% | | | (34.9) |
| 4 | GMO 10% | (none) | (none) | 23.7 |
| (73.3) | DPG 10% | | | (6.1) |
| 5 | GMO 10% | 10% | (none) | 78.1 |
| (73.3) | DPG 10% | crospovidone | | (15.9) |
| 6 | GMO 10% | 10% PVP | (none) | 37.2 |
| (73.3) | DPG 10% | | | (2.0) |
| 7 | GMO 10% | (none) | Silicone | 21.6 |
| (73.3) | DPG 10% | | Blend A | (4.7) |
| 8 | GMO 10% | 10% | Silicone | 67.3 |
| (73.3) | DPG 10% | crospovidone | Blend A | (4.5) |
| 9 | GMO 10% | 10% PVP | Silicone | 40.7 |
| (73.3) | DPG 10% | | Blend A | (5.9) |
| 10 | GMO 10% | (none) | Silcone | 28.9 |
| (83.3) | MeLau 5% | | Blend B | (2.4) |
| 11 | GMO 10% | 10% | Silcone | 92.2 |
| (73.3) | MeLau 5% | crospovidone | Blend B | (8.7) |
| 12 | GMO 10% | 10% PVP | Silcone | 200.1 |
| (73.3) | MeLau 5% | | Blend B | (16.4) |

GMO = glyceryl monooleate
MeLau = methyl laurate
DPG = dipropylene glycol
% = % by weight based on the wet weight of the polymer matrix

As seen from the shear data, matrices formulated without a humectant exhibited very low shear (and low cohesive strength), while matrices formulated with a humectant exhibited satisfactory shear values (and satisfactory cohesive strength). As seen in the figures, matrices formulated with a humectant also exhibited good drug flux.

The results surprisingly show that systems as described herein comprising various combinations of GMO, DPG and MeLau enhancers can achieve good drug flux with good Jmax values and also achieve sustained drug flux over a prolonged delivery period, achieving increased cumulative delivery with a relatively low drop-off See, e.g., FIGS. 2-5.

The results also surprisingly show that the use of a face adhesive did not substantially impact drug flux. See FIGS. 6 and 7. Furthermore, the shear values of the systems with a face adhesive were comparable to that of corresponding systems without a face adhesive.

### EXAMPLE 2

Polymer matrices comprising 0.5% ethinyl estradiol and 1.2% levonogestrel in a hydroxy-functional acrylic polymer were prepared with the following components, applied to a backing material, dried, and optionally provided a face adhesive layer comprising a silicone pressure-sensitive adhesive. Drug flux through human cadaver skin was assessed, and compared to Climara® Pro and another reference system having DMSO as an enhancer. Results are set forth in the tables below.

| Formulation | | | Face |
|---|---|---|---|
| (wt % acrylic polymer) | Enhancers | Humectant | Adhesive |
| Climara | (none) | copovidone | (none) |
| Pro ® | | | |
| Reference | 9.5% DMSO | 10% | (none) |
| Composition | 3.1% LL | crospovidone | |
| | 3.1% EL | | |
| | 2.39% CA | | |
| 13 | GMO 10% | 10% PVP | (none) |
| (68.3) | DPG 10% | | |
| 14 | GMO 10% | 10% PVP | Yes |
| (68.3) | DPG 10% | | |
| 15 | GMO 10% | 5% PVP | (none) |
| (65.3) | DPG 10% | | |
| | Transcutol ® 8% | | |
| 16 | GMO 10% | 3.34% PVP | Yes |
| (66.96) | DPG 10% | | |
| | Transcutol ® 8% | | |
| 17 | GMO 10% | 5% PVP | Yes |
| (65.3) | DPG 10% | | |
| | Transcutol ® 8% | | |
| 18 | GMO 10% | 5% PVP | (none) |
| (65.3) | DPG 10% | | |
| | DMI 8% | | |
| 19 | GMO 10% | 5% PVP | Yes |
| (65.3) | DPG 10% | | |
| | DMI 8% | | |

Reference Composition: 76.86% hydroxy-functional acrylic polymer; 9.5% dimethyl sulfoxide (DMSO); 3.1% lauryl lactate (LL); 3.1% ethyl lactate (EL); and 2.39% capric acid (CA).
GMO = glyceryl monooleate
DPG = dipropylene glycol
Transcutol ® = diethylene glycol monoethyl ether
DMI = dimethyl isosorbide
% = % by weight based on the wet weight of the polymer matrix

Levonorgestrel Flux

| | | LNG | LNG | LNG | |
|---|---|---|---|---|---|
| Formulation | LNG | Jₘₐₓ Ratio | Avg. | Cum. Flux | LNG |
| (wt % acrylic | Avg. | to | Cum. | Ratio to | Avg. |
| polymer) | Jₘₐₓ | Reference | Flux | Reference | Drop |
| Climara | 0.029 | 0.29 | 3.45 | 0.5 | 59.04 |
| Pro ® | | | | | |
| Reference | .101 | 1.00 | 7.07 | 1.00 | 81.26 |
| Composition | | | | | |
| 13 | .076 | 0.75 | 7.69 | 1.09 | 64.63 |
| (68.3) | | | | | |
| 14 | .056 | 0.55 | 7.64 | 1.09 | 36.61 |
| (68.3) | | | | | |
| 15 | .113 | 1.12. | 11.12 | 1.59 | 67.14 |
| (65.3) | | | | | |
| 16 | .078 | 0.77 | 10.68 | 1.53 | 39.61 |
| (66.96) | | | | | |
| 17 | .062 | 0.61 | 7.89 | 1.10 | 41.26 |
| (65.3) | | | | | |
| 18 | .097. . . | 0.96 | 10.42 | 1.47 | 63.06 |
| (65.3) | | | | | |
| 19 | .071 | 0.70 | 9.26 | 1.32 | 48.79 |
| (65.3) | | | | | |

Ethinyl Estradiol Flux

| | | | | EE | |
|---|---|---|---|---|---|
| Formulation | | EE | EE | Avg. | |
| (wt % | EE | Avg. Jₘₐₓ | Avg. | Cum. Flux | EE |
| acrylic | Avg. | Ratio to | Cum. | Ratio to | Avg. |
| polymer) | Jₘₐₓ | Reference | Flux | Reference | Drop |
| Climara | 0.064 | 2.6 | 7.33 | 2.58 | 61.52 |
| Pro ® | | | | | |
| Reference | .025 | 1.00 | 2.90 | 1.00 | 34.29 |
| Composition | | | | | |
| 13 (68.3) | .029 | 1.19 | 3.53 | 1.23 | 41.96 |
| 14 (68.3) | .031 | 1.27 | 3.30 | 1.15 | 0.00 |
| 15 (65.3) | .037 | 1.50 | 4.28 | 1.50 | 30.58 |
| 16 (66.96) | .032 | 1.31 | 3.78 | 1.33 | 0.00 |
| 17 (65.3) | .028 | 1.15 | 2.67 | 0.92 | 36.71 |
| 18 (65.3) | .034 | 1.37 | 3.74 | 1.33 | 65.37 |
| 19 (65.3) | .024 | 0.98 | 2.82 | 1.00 | 65.16 |

The results surprisingly show that systems as described herein comprising GMO and DPG enhancers can achieve drug flux comparable to that of the reference DMSO-based composition (as shown by the “ratio” data reported above), which is reported to be effective for contraception. The results also surprisingly show that systems as described herein comprising GMO, DPG and diethylene glycol monoethyl ether or DMI as enhancers can achieve flux levels greater than the reference DMSO-based composition.

As with Example 1, these results also show that the face adhesive does not negatively impact drug flux, which was surprising.

### EXAMPLE 3

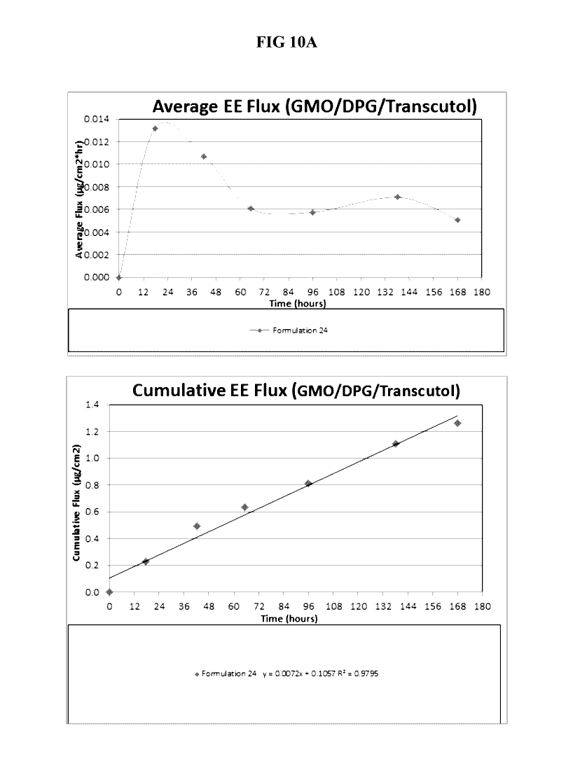

Polymer matrices comprising 0.3% ethinyl estradiol and 1.0% levonogestrel in a hydroxy-functional acrylic polymer were prepared with the following components, applied to a backing layer, and dried. Drug flux through human cadaver skin was assessed, and compared to Climara® Pro and another reference system having DMSO as an enhancer. Results are set forth in the tables below and in FIG. 8.

Formulation 20 (% by weight dry):

EE=0.3%

LNG=1.0%

PVP K-30=5.0%

GMO=3.0%

DPG=4.25%

Transcutol®=0.3%

DuroTak® 87-2516=Q.S. (˜86.15%)

Formulation 21 (% by weight dry):

EE=0.3%

LNG=1.0%

PVP K-30=5.0%

GMO=3.0%

DPG=2.75%

Transcutol®=0.4%

DuroTak® 87-2516=Q.S. (˜87.55%)

Formulation 22(% by weight dry):

EE=0.3%

LNG=1.0%

PVP K-30=5.0%

GMO=3.0%

DPG=2.75%

DMI=2.5%

DuroTak® 87-2516=Q.S. (85.45%)

Levonorgestrel Flux

| | | | | LNG | |
|---|---|---|---|---|---|
| | | | | Cum. | |
| | | LNG | LNG | Flux | |
| Formulation | LNG | Jₘₐₓ Ratio | Avg. | Ratio | LNGA |
| (wt % acrylic | Avg. | to | Cum. | to | vg. |
| polymer) | Jₘₐₓ | Reference | Flux | Reference | Drop |
| Climara | .024 | 0.30 | 2.16 | 0.48 | 82.49 |
| Pro ® | | | | | |
| Reference | .080 | 1.00 | 4.52 | 1.00 | 98.86 |
| Composition | | | | | |
| 20 | .052 | 0.65 | 5.74 | 1.27 | 66.16 |
| (86.15) | | | | | |
| 21 | .066 | 0.83 | 7.36 | 1.63 | 60.07 |
| (87.55) | | | | | |
| 22 | .047 | 0.59 | 5.12 | 1.13 | 65.56 |
| (85.45) | | | | | |

Ethinyl Estradiol Flux

| | | | | EE | |
|---|---|---|---|---|---|
| | | EE | | Avg. | |
| | EE | Avg. Jₘₐₓ | EE Avg. | Cum. Flux | EE |
| | Avg. | Ratio to | Cum. | Ratio to | Avg. |
| Formulation | Jₘₐₓ | Reference | Flux | Reference | Drop |
| Climara | .093 | 1.69 | 7.99 | 4.14 | 70.96 |
| Pro ® | | | | | |
| Reference | .055 | 1.00 | 1.93 | 1.00 | 94.73 |
| Composition | | | | | |
| 20 | .023 | 0.42 | 1.36 | 0.70 | 79.59 |
| (86.15) | | | | | |
| 21 | .026 | 0.47 | 1.69 | 0.88 | 76.54 |
| (87.55) | | | | | |
| 22 | .014 | 0.25 | 1.27 | 0.66 | 61.70 |
| (85.45) | | | | | |

### EXAMPLE 4

Polymer matrices were prepared to maximize levonogestrel content based on solubility and drug flux, and ethinyl estradiol content was varied to obtain a ratio of ethinyl estradiol to levonogestrel suitable for use for contraception. Matrices were applied to a backing layer and release liner and their properties were assessed as summarized below.

Formulation 23 (% by weight drv)

EE=0.25%

LNG=1.0%

IPM=5%

DPG=5%

Transcutol®=0.3%

K-30=10%

DuroTak 87-2516=Q.S.=78.45%

Applied at a coat weight of 12.5 mg/cm²

Silicone Pressure-Sensitive Adhesive Face Adhesive (5 mg/cm²)

Formulation 24 (% by weight dry)

EE=0.3%

LNG=1.0%

GMO=6.5%

DPG=2.75%

Transcutol®=0.4%

K-30=5%

DuroTak 87-2516=Q.S.=84.05%

Coat Weight: 12.5 mg/cm²

Formulation 25 (% by weight dry)

EE=0.3%

LNG=1.0%

GMO=3%

DPG=2.75%

Transcutol®=0.4%

K-30=5%

Bio PSA 4502=15%

DuroTak 87-2516=Q.S.=72.55%

Coat Weight: 12.5 mg/cm²

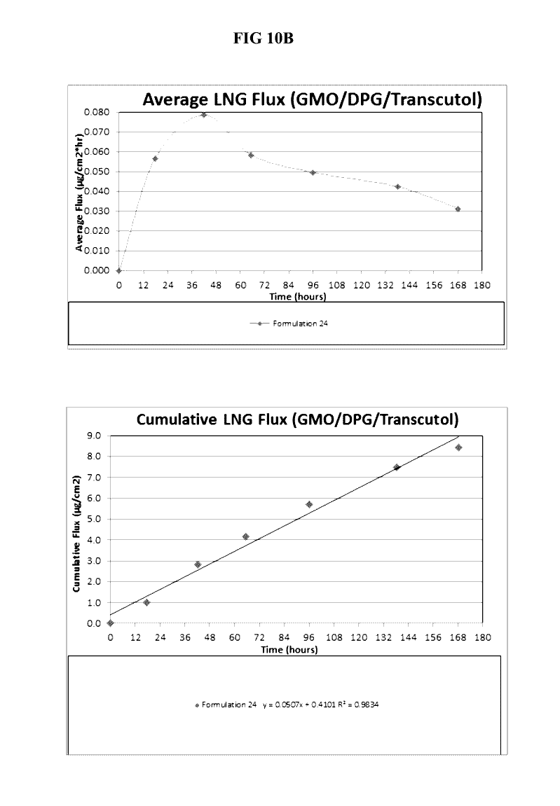

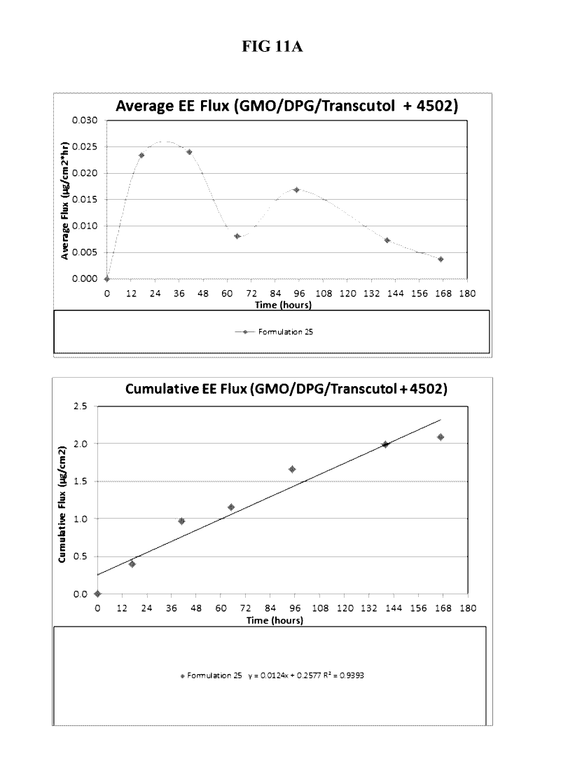

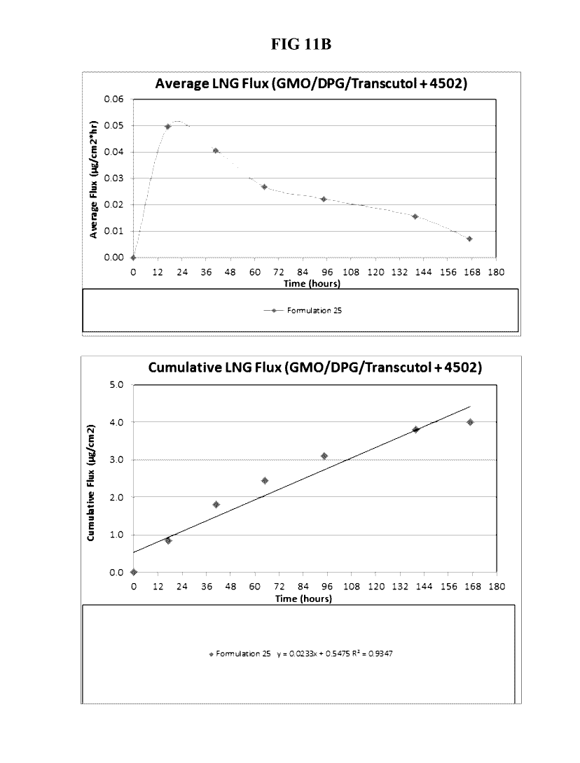

FIGS. 9-11 illustrate average and cumulative ethinyl estradiol (EE) (FIGS. 9A, 10A, 11A) and levonorgestrel (LNG) (FIGS. 9B, 10B, 11B) flux (μg/cm²/hr) over time (0-168 hours) from transdermal delivery systems described in Example 4 (Formulations 23-25). The results show that the systems as described herein comprising the enhancers dipropylene glycol, diethylene glycol monoethyl ether, and either glyceryl monooleate or isopropyl myristate, can achieve good drug flux and also achieve sustained drug flux over a prolonged delivery period.

Placebo versions of formulations 23-25 (without the EE and LNG) performed well in wear studies and animal irritation studies (data not shown), making them particularly good formulations for use in 7-day applications.

The results of these studies also show that systems as described herein comprising a polymer matrix that includes isopropyl myristate as an enhancer and a silicone pressure-sensitive adhesive face adhesive exhibit both good flux properties and particularly good 7-day wear properties, exhibiting superior adhesion in 7-day wear studies.

What is claimed is:

1. A transdermal drug delivery system for the transdermal delivery of levonorgestrel and ethinyl estradiol in the form of a flexible finite system for topical application, comprising a polymer matrix comprising levonorgestrel, ethinyl estradiol, an acrylic polymer that comprises a hydroxy functional acrylic polymer, a humectant, a first enhancer selected from the group consisting of glyceryl monooleate, dipropylene glycol, and methyl laurate, a second enhancer different from the first enhancer and selected from the group consisting of glyceryl monooleate, dipropylene glycol, methyl laurate, diethylene glycol monoethyl ether, and dimethyl isosorbide, and, optionally, and a third enhancer different from the first and second enhancers and selected from the group consisting of glyceryl monooleate, dipropylene glycol, methyl laurate, diethylene glycol monoethyl ether, and dimethyl isosorbide.

2. The transdermal drug delivery system of claim 1, wherein the polymer matrix comprises on a % dry weight basis about 0.1-3% levonorgestrel, about 0.1-5% ethinyl estradiol, about 1-30% of the first, second and optional third penetration enhancers, about 3-10% humectant, and the balance acrylic polymer(s).

3. The transdermal drug delivery system of claim 1, wherein the humectant is selected from the group consisting of polyvinylpyrrolidone (PVP), crosslinked PVP (crospovidone) and polyvinylpyrrolidone/vinylacetate (copovidone).

4. The transdermal drug delivery system of claim 1, wherein the first and second enhancers are dipropylene glycol and diethylene glycol monoethyl ether.

5. The transdermal drug delivery system of claim 1, wherein the first, second and third enhancers are dipropylene glycol, diethylene glycol monoethyl ether, and glyceryl monooleate.

6. The transdermal drug delivery system of claim 1, wherein the first and second enhancers are dipropylene glycol and diethylene glycol monoethyl ether, and the composition further comprises isopropyl myristate as a third enhancer .

7. The transdermal drug delivery system of claim 1, wherein the polymer matrix further comprises a silicone pressure-sensitive adhesive.

8. The transdermal drug delivery system of claim 1, comprising an amount of levonorgestrel sufficient to achieve sustained delivery of levonorgestrel over a period of time of at least 3 days, at least 4 days, or at least 7 days.

9. The transdermal drug delivery system of claim 1, comprising an amount of ethinyl estradiol sufficient to achieve sustained delivery of ethinyl estradiol over a period of time of at least 3 days, at least 4 days, or at least 7 days.

10. The transdermal drug delivery system of claim 1, further comprising a backing layer.

11. The transdermal drug delivery system of claim 1, further comprising a release liner.

12. The transdermal drug delivery system of claim 1, further comprising a face adhesive layer disposed on a skin-contacting side of the polymer matrix.

13. The transdermal drug delivery system of claim 12, wherein face adhesive layer comprises a silicone pressure-sensitive adhesive.

14. The transdermal drug delivery system of claim 13, wherein the polymer matrix further comprises isopropyl myristate as a third enhancer.

15. A method of transdermally delivering levonorgestrel and ethinyl estradiol comprising applying a transdermal drug delivery system according to claim 1 to the skin or mucosa of a subject in need thereof, wherein the transdermal drug delivery system optionally comprises a face adhesive layer disposed on a skin-contacting side of the polymer matrix.

16. The method of claim 15, wherein the subject is a human female subject and the method is for contraception.

17. The method of claim 15, wherein the transdermal drug delivery system is applied for a duration of up to 7 days.

18. A method of preparing a transdermal drug delivery system comprising levonorgestrel and ethinyl estradiol in the form of a flexible finite system for topical application, comprising preparing a polymer matrix comprising levonorgestrel, ethinyl estradiol, a hydroxyl functional acrylic polymer, a humectant, a first enhancer selected from the group consisting of glyceryl monooleate, dipropylene glycol, and methyl laurate, a second enhancer different from the first enhancer and selected from the group consisting of glyceryl monooleate, dipropylene glycol, methyl laurate, and, optionally, a third enhancer different from the first and second enhancers and selected from the group consisting of glyceryl monooleate, dipropylene glycol, methyl laurate and isopropyl myristate.

19. The method of claim 18, further comprising applying a face adhesive layer to the polymer matrix.
